(19) United States
(12) Patent Application Publication
(10) Pub. No.: US 2022/0233489 A1
(43) Pub. Date: Jul. 28, 2022

(54) KITS FOR DERMAL CARE

(71) Applicant: ChemistryRX, Folcroft, PA (US)

(72) Inventors: Amany Mansour-Awad, Philadelphia, PA (US); Lars Brichta, Brooklyn, NY (US)

(21) Appl. No.: 17/587,307

(22) Filed: Jan. 28, 2022

Related U.S. Application Data
(60) Provisional application No. 63/199,847, filed on Jan. 28, 2021.

(51) Int. Cl. A61K 31/203; A61K 8/67; A61K 9/00; A61Q 19/08; A61K 31/07; A61K 31/11; A61K 31/025; A61K 31/216; A61K 31/728; A61K 31/455; A61K 31/197

(52) U.S. Cl. CPC A61K 31/203; A61K 8/671; A61K 9/0014; A61Q 19/08; A61K 31/07; A61K 31/197; A61K 31/025; A61K 31/216; A61K 31/728; A61K 31/455; A61K 31/11

(57) ABSTRACT

Compositions and methods for treating administering retinoid or retinoid-related compounds without side effects.

### CROSS-REFERENCE TO RELATED APPLICATIONS

This application claims priority from U.S. Provisional No. 63/199,847, entitled “Kits for Dermal Care,” filed on Jan. 28, 2021, the entirety of which is hereby incorporated by reference.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Retinoids and retinoid-related compounds have important and diverse functions in a biological system and have been used to treat various skin conditions such as acne, photoaging, psoriasis, ichthyosis, hair loss, and tumors. However, time and/or dosage related adverse effects have been also reported such redness, swelling, and/or itching of skin, swellings on the long bones, anorexia, skin lesions, hair loss, hepatosplenomegaly, papilledema, bleeding, general malaise, pseudotumor cerebri, and even death. Without wishing to be bound by theory, the compositions and methods of embodiments may allow patients to achieve topical application of therapeutic concentrations of retinoid or retinoid-related compounds without the side effects commonly associated with such treatment.

### SUMMARY OF THE INVENTION

### Description of the Drawings

Not applicable

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, 2 ml, 3 ml, 4 ml, 5 ml, 6 ml, and 7 ml are also intended to be explicitly disclosed, as well as the range of values greater than or equal to 1 ml and the range of values less than or equal to 8 ml.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering,” or “administration” as used herein refer to either directly administering a compound or composition (also referred to as an agent of interest) or pharmaceutically acceptable salt of the compound (agent of interest) or a composition to a subject or providing a compound or composition to the patient and allowing the patient to self administer the compound or composition.

The term “carrier” as used herein encompasses carriers, excipients, and diluents, meaning a material, composition, or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance, reduce, normalize, or adjust the growth, texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government or listed in the U.S. Pharmacopeia or other generally recognized pharmacopeia for use in mammals (e.g. animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is an adult or child human.

The term “treating” is used herein, for instance, in reference to methods of treating a disorder or a condition, and generally includes the administration of a compound or composition which reduces the frequency of, or delays the onset of, symptoms of a medical condition or enhance, reduce, normalize or adjust the growth, texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

As used herein, the term “therapeutic” means an agent utilized to treat, combat, ameliorate, prevent or improve an unwanted condition or disease of a subject.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entirety are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed methods for treating skin, compositions associated with such methods, and kits containing the compositions. In general, such methods may include the steps of topically administering to the skin of a patient in need of treatment a series of compositions containing increasing amounts of a retinoid or retinoid-related compound over the course of treatment. In some embodiments, each composition containing a specific concentration of retinoid or retinoid-related compound may be administered for one day to two weeks, before administration of another composition with an increased amount of retinoid or retinoid-related compound. The compositions may include retinoid or a retinoid-related compound, a topically acceptable excipient, carrier, or diluent, and one or more additional components that aid in treatment of the individual, mitigate side effects of topical retinoid or retinoid-related compound administration or combinations thereof, and kits may provide a course of treatment including two or more compositions associated with the methods and instructions for carrying out the methods of the invention.

The methods, compositions, and kits of various embodiments may include one or more retinoid or retinoid-related compounds. Retinoids are a class of compounds that include a four-isoprenoid unit in their molecular structures. Examples of retinoids include retinol (vitamin A), retinal, retiferol, tretinoin (all-trans-retinoic acid, e.g. retinoic acid, Retin-A), isotretinoin, alitretinoin (9-cis-retinoic acid), etretinate, acitretin, tazarotene, bexarotene and Adapalene. Retinoids and retinoid-related compounds have important and diverse functions in a biological system and have been used to treat various skin conditions such as acne, photoaging, psoriasis, ichthyosis, hair loss, and tumors. However, time and/or dosage related adverse effects have been also reported such redness, swelling, and/or itching of skin, swellings on the long bones, anorexia, skin lesions, hair loss, hepatosplenomegaly, papilledema, bleeding, general malaise, pseudotumor cerebri, and even death. Without wishing to be bound by theory, the compositions and methods of embodiments may allow patients to achieve topical application of therapeutic concentrations of retinoid or retinoid-related compounds without the side effects commonly associated with such treatment.

Certain embodiments may include a maximum concentration of retinoid or retinoid-related compound of about 1.0 wt. % to about 1.5 wt. %, which is achieved following administration of compositions having concentrations of retinoid or retinoid-related compound of about 10%, about 25%, about 50%, about 75% of the maximum concentration or any individual percentage encompassed by these example concentrations for 1 to 14 days, 1 to 4 or 6 weeks or monthly for 1 to 4 or 6 months, individually. For example, in some embodiments, the methods may include administering, daily or weekly, a composition having concentrations of retinoid or retinoid-related compound of about 10% of a maximum concentration for 1 to 14 days or 1 to 4 or 6 weeks, followed by administering, daily or weekly, a composition having concentrations of retinoid or retinoid-related compound of about 5% to about 25% of a maximum concentration for 1 to 14 days or 1 to 4 or 6 weeks, followed by administering, daily or weekly, a composition having concentrations of retinoid or retinoid-related compound of about 30% to about 50% of a maximum concentration for 1 to 14 days or 1 to 4 or 6 weeks, followed by administering, daily or weekly, a composition having concentrations of retinoid or retinoid-related compound of about 70% to about 90% of a maximum concentration for 1 to 14 days or 1 to 4 or 6 weeks, followed by administering, daily or weekly, a composition having concentrations of retinoid or retinoid-related compound of about 100% of a maximum concentration of about 1.0 wt. % to about 1.5 wt. % until a therapeutically effective or cosmetically practical endpoint is reached. For example, the patient may continue administering, daily or weekly, a composition having concentrations of retinoid or retinoid-related compound of about 100% of a maximum concentration of about 1.0 wt. % to about 1.5 wt. %, for weeks, months, years, or decades.

In particular embodiments, the methods may include the step of administering to a first composition containing less than about 0.5 wt. % retinoid or retinoid-related compounds and administering at least a second composition containing more retinoid or retinoid-related compounds than the first composition and less than about 1.5 wt. % retinoid or retinoid-related compounds. For example, in some embodiments, the method may include administering a first composition containing about 0.08 wt. % to about 0.5 wt. % retinoid or retinoid-related compound and at least a second composition that includes more retinoid or retinoid-related compound and contains about 0.3 wt. % to about 1.5 wt. % retinoid or retinoid-related compounds.

In further embodiments, one more additional compositions may be administered during the course of treatment. For example, the methods of embodiments may include administering a first composition containing 0.08 wt. % to about 0.5 wt. % retinoid or retinoid-related compound, followed by administrating a second composition containing about 0.3 to about 0.8 wt. % retinoid or retinoid-related compound, followed by administrating a third composition containing about 0.5 wt. % to about 1.5 wt. % retinoid or retinoid-related compound. In other embodiments, the methods may include administering a first composition containing 0.08 wt. % to about 0.5 wt. % retinoid or retinoid-related compound, followed by administrating a second composition containing about 0.3 to about 0.8 wt. % retinoid or retinoid-related compound, followed by administrating a third composition containing about 0.5 wt. % to about 0.9 wt. % retinoid or retinoid-related compound, and administering a fourth composition containing about 0.7 wt. % to about 1.5 wt. % retinoid or retinoid related compounds. The methods may include any number of compositions that gradually increase in concentration of retinoid or retinoid-related compound, and thus, any number of administering steps.

In some embodiments, each composition may individually be administered to the patient daily for one to 14 days. For example, the methods of embodiments may include administering a first composition containing 0.08 wt. % to about 0.5 wt. % retinoid or retinoid-related compound for 1 to 14 days, followed by administrating a second composition containing about 0.3 to about 0.8 wt. % retinoid or retinoid-related compound for 1 to 14 days, followed by administrating a third composition containing about 0.5 wt. % to about 1.5 wt. % retinoid or retinoid-related compound for 1 to 14 days. In other embodiments, the methods may include administering a first composition containing 0.08 wt. % to about 0.5 wt. % retinoid or retinoid-related compound for 1 to 14 days, followed by administrating a second composition containing about 0.3 to about 0.8 wt. % retinoid or retinoid-related compound for 1 to 14 days, followed by administrating a third composition containing about 0.5 wt. % to about 0.9 wt. % retinoid or retinoid-related compound for 1 to 14 days, followed by administering a fourth composition containing about 0.7 wt. % to about 1.5 wt. % retinoid or retinoid related compounds for 1 to 14 days.

In some embodiments, each composition may individually be administered to the patient weekly for 1 to 4 or 6 weeks or monthly for 1 to 4 or 6 months. Alternatively, each composition may be individually administered until the patient is substantially free from side effects. For example, methods include administering a first composition containing 0.08 wt. % to about 0.5 wt. % retinoid or retinoid-related compound to a patient until administering does not result in redness or itching of the skin, followed by administrating a second composition containing about 0.3 to about 0.8 wt. % retinoid or retinoid-related compound to a patient until administering does not result in redness or itching of the skin, followed by administrating a third composition containing about 0.5 wt. % to about 1.5 wt. % retinoid or retinoid-related compound to a patient until administering does not result in redness or itching of the skin. The patient may therefore determine an appropriate course of treatment based on the patient's physiological response to the compositions.

In some embodiments, the compositions may include various additional components that may improve the dermatological acceptability of topically administering relatively high concentrations of retinoid or retinoid-related compounds. For example, in some embodiments, the compositions may include a glycosaminoglycan, polyglycols, polyanionic polysaccharides, nitrogen containing polymers, vitamin B₃ related compounds, dexpanthenol, and the like and combinations thereof. Such compositions may reduce side effects associated with topically administering retinoid or retinoid-related compound containing compositions, which may allow for increasing the concentration of retinoid or retinoid-related compound in low dose compositions, for example, a first compositions may include about 20% of a maximum concentration of retinoid or retinoid-related compound instead of about 5% of the maximum concentration, reducing the time period between administering compositions containing gradually increasing concentrations of retinoid or retinoid-related compound, for example, from 4 to 6 weeks to 1 to 2 weeks, or both.

In some embodiment, the compositions may include a glycosaminoglycan such as, for example, hyaluronan, hyaluronic acid, hyaluronate, sodium hyaluronate, dermatan sulfate, karatan sulfate, chondroitin 6-sulfate, heparin, and the like, and combinations thereof; polyglycols such as polyethylene glycol and the like and combinations thereof; polyanionic polysaccharides such as dextran, sodium alginate, alginic acid, propylene glycol alginate, carboxymethyl cellulose, carboxyethyl cellulose, hydroxyl ethyl cellulose, hydroxyl propyl methyl cellulose, hydroxy propyl ethyl cellulose, hydroxy propyl cellulose, methyl cellulose, polylysine, polyhistidine, polyhydroxy proline, poly ornithine, polyvinyl pyrolidone, polyvinyl alcohol, chitosan, and the like and combinations thereof; long chain nitrogen containing polymers such as polylysine, polyvinylpyrrolidone, polyvinyl alcohol, and the like and combinations thereof. In certain embodiments, the compositions may include a combination glycosaminoglycans, polyglycols, polyanionic polysaccharides, and nitrogen containing polymers, and in particular embodiments, the compositions may include hyaluronan, hyaluronic acid, hyaluronate, sodium hyaluronate, or combinations thereof.

The concentration of glycosaminoglycans, polyglycols, polyanionic polysaccharides, and nitrogen containing polymers in the compositions of the invention may vary among embodiments. For example, these components may each individually be present in a concentration of about 0.01 wt. % to about 1.0 wt. %, about 0.05 wt. % to about 1.0 wt. %, about 0.1 wt. % to about 0.9 wt. %, about 0.1 wt. % to about 0.8 wt. %, or any range or individual concentration encompassed by these example ranges.

In some embodiments, the compositions may include one or more vitamin B₃ compounds such as niacin, niacinamide, nicotinic acid, nicotinyl alcohol, nicotinic acid esters, nicotinyl amino acids, nicotinyl alcohol esters of carboxylic acids, nicotinic acid N-oxide and niacinamide N-oxide, tocopherol nicotinate, inositol hexanicotinate, 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, and niaprazine, and the like and derivatives, salts or combinations thereof.

The concentration of vitamin B₃ compounds in the compositions of the invention may vary among embodiments. For example, each vitamin B₃ compound may individually be present in a concentration of about 0.01 wt. % to about 1.0 wt. %, about 0.05 wt. % to about 1.0 wt. %, about 0.1 wt. % to about 0.9 wt. %, about 0.1 wt. % to about 0.8 wt. %, or any range or individual concentration encompassed by these example ranges.

In some embodiments, the compositions may include dexpanthenol, the concentration of which may vary among embodiments. For example, dexpanthenol may be present in a concentration of about 0.01 wt. % to about 1.0 wt. %, about 0.05 wt. % to about 1.0 wt. %, about 0.1 wt. % to about 0.9 wt. %, about 0.1 wt. % to about 0.8 wt. %, or any range or individual concentration encompassed by these example ranges.

The compositions of various embodiments are formulated as topical compositions and may, generally, include a base. Such bases include, for example, white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, and the like and combinations thereof.

In some embodiments, the base may be a liposomal base. Liposomal bases are an emulsion that includes a lipophilic component and an aqueous component that can be in the form of a lotion, a cream, a gel, or a paste. Examples of suitable liposomal bases include PCCA Lipoderm®, Lipoderm ActiveMax™, Anhydrous Lipoderm, and Lipoderm High Molecular Weight™ PCCA. Such liposomal base formulations can include, for example, about 60-80% wt/wt water combined with glycerin, C₁₂₋₁₅ alkyl benzoate, glyceryl stearate, dimethicone, cetearyl alcohol, cetearyl glucoside, polyacrylamide, cetyl alcohol, magnesium aluminum silicate, xanthan gum, aloe vera (aloe barbadensis), tocopheryl acetate (vitamin E acetate), prunus amygadalus amara (bitter almond) kernel oil, Vitis vinifera (Grape) seed extract, Triticum vulgare (wheat) germ oil, retinyl palmitate (vitamin A palmitate), ascorbyl palmitate (vitamin C palmitate), Pro-Lipo Multi-emulsion Liposomic System, tetrasodium EDTA, phenoxyethanol, sodium hydroxymethylglycinate and the like and combinations thereof.

In some embodiments, the base may be cream base. Cream bases are semi-solid emulsions of oil and water. They are divided into two types: oil-in-water (O/W) creams which are composed of small droplets of oil dispersed in a continuous water phase, and water-in-oil (W/O) creams which are composed of small droplets of water dispersed in a continuous oily phase. Oil-in-water creams are more comfortable and cosmetically acceptable as they are less greasy and more easily washed off using water. Water-in-oil creams are more difficult to handle but many drugs which are incorporated into creams are hydrophobic and will be released more readily from a water-in-oil cream than an oil-in-water cream. Water-in-oil creams are also more moisturising as they provide an oily barrier which reduces water loss from the stratum corneum, the outermost layer of the skin. Cream bases typically include water, oil, emulsifier, and thickening agents, such as those discussed below.

In some embodiments, the base may be a moisturizing cream base. Moisturizing cream bases are composed of the same components as the cream bases described above with the addition of an emollient or humectant, that may provide a barrier that reduces water loss from the stratum corneum, the outermost layer of the skin. The emollient or humectant in a moisturizing cream base may be cetyl esters wax, stearyl alcohol, cetyl alcohol, and glycerin, or combinations thereof.

Example cream bases and moisturizing cream bases include VersaBase (PCCA); Emollient cream, Vanishing cream, CeraVe, Vanicream, Vitamin E; Cliniderm; Dermabase (purified water, petrolatum, mineral oil, cetostearyl alcohol); Eucerin (water, petrolatum, mineral oil, ceresin, lanolin alcohol, methylchloroisothiozolinone, methylisothiazolinone); Glaxal (WellSpring Pharmaceutical Corp., Sarasota, Fla.); stearic acid cream, or any other pharmaceutical cream base used for topical formulations known to those skilled in the art.

In some embodiments, the base may be an ointment base. Ointments are compositions in which oil and water are provided in a ratio of from 7:1 to 2:1, from 5:1 to 3:1, or 4:1, and in some embodiments, the ointment may or may not include water, such as Aquaphor, Pracasil, and plasticized bases. Ointments are generally formulated using oils, waxes, water, alcohols, petroleum products, silicones, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

The amount of base in the compositions of embodiments can vary and will depend on the amounts of the other components. More base can be added to compensate for smaller amounts of other components in the desired topical pharmaceutical formulation. In some embodiments, the base may be present in a concentration of about 45% (w/w) to about 99.75% (w/w) of the total composition, or any range or individual concentration known in the art.

Certain embodiments the compositions containing a retinoid or retinoid-related compound may be formulated as a film forming composition. Such film forming compositions, when dried, form a film over the site of application. An example of a film forming composition that is suitable for use in this invention is Flexible Collodion, USP. As described in Remington: The Science and Practice of Pharmacy, 19th Ed. (Easton, Pa.: Mack Publishing Co., 1995). Collodions are ethyl ether/ethanol solutions containing pyroxylin (a nitrocellulose) that evaporate to leave a film of pyroxylin. In some embodiments, the film former may act as a carrier.

In some embodiments, the film forming compositions may include a polymer or mixture of polymers that when applied to skin form a more resilient barrier than typical collodion compositions. Polymers that can be used in such embodiments include, for example, polymers or copolymers of acrylic acid (PAA), polymers or copolymers of methacrylic acid (PMA), polymers or copolymers of itaconic acid (PIA), polymers or copolymers of maleic acid (PLA), polyalkylenes, and polymers or copolymers of 3-butene-1,2,3-tricarboxylic acid (PBA), or combinations thereof. Polyalkylenes include, for example, polybutylene and polyisoprene. The polymers of embodiments can have an average molecular weight of about 300 kD to about 2000 kD and may have a viscosity index of about 70 mm²/s to about 122 mm²/s (ASTM D2270).

Polyacrylic acid homopolymers and polyacrylate encompass a wide variety of well known compounds. For example, polyacrylate homopolymers include polymers of acrylic acid and an alkyl acrylate, polymers of acrylamide and acrylic acid, polymers of acrylamide and an alkyl acrylate, polymers of alkyl acrylates and methacrylic acid, copolymers of ethyl methacrylate, abietyl methacrylate, and diethylaminoethyl methacrylate, which can be quaternized with dimethyl sulfate, polymers of ethyl methacrylate, oleyl methacrylate, and diethylaminoethyl methacrylate, which can be quaternized with dimethyl sulfate, and the like and salts thereof. Polyacrylate copolymers include, for example, copolymers of alkyl acrylates and ethylene, copolymers of acrylic acid and vinyl alcohol, and the like and salts thereof. Particular examples of polyacrylate copolymers include poly(methyl methacrylate).

In such embodiments, the amount of polymer may be about 0.05 wt. % to about 30 wt. % of the total composition, and in some embodiments, the concentration of hydrophilic polymer may be about 0.5 wt. % to about 10 wt. %, about 1.0 wt. % to about 7.5 wt. %, about 5 wt. % to about 10 wt. %, or any range or individual concentration encompassed by these example ranges. The concentration of crosslinking agent may be about 0.5 wt. % to about 10 wt. %, about 2 wt. % to about 8 wt. % or any range or individual concentration encompassed by these example ranges. In particular embodiments, the polymer may be a polyalkylene which can be provided in an amount of about 0.05 wt. % to about 5 wt. %, about 0.1 wt. % to about 1 wt. % of the total composition, or any individual concentration or range encompassed by these example ranges. In embodiments in which the polymer is a polyacrylate homopolymer or a polyacrylate copolymer, the polymer may be about 0.05 wt. % to about 10 wt. %, about 0.1 wt. % to about 1 wt. % of the total composition, or any individual concentration or range encompassed by these example ranges.

In some embodiments, the film forming compositions may further include crosslinking agents, plasticizers, and the like and combinations thereof. Crosslinking agent include, for example, polyvinyl alcohol (PVA), polyvinyl pyrrolidone (PVP), polyethylene oxide (PEO), copolymer combinations of propylene oxide and ethylene oxide [P(EO/PO)], and gelatin. Any plasticizer may be used in the compositions of the invention. Example plasticizers include, for example, propylene glycol, polyhydroxy compounds of low molecular weight including glycerol, sorbitol, gluconolactone, gluconic acid, urea, or combinations thereof. Embodiments include commercially available film forming agents, such as Occlusaderm, which consists of, water, pullulan, glycerin, glyceryl, polyacrylate, PEG-90M disodium EDTA, sodium dihydroacetate, and methylisothiazolinone.

In some embodiments, the compositions may be formulated as a mask. Such compositions may include retinoid or retinoid-related compounds and, in some embodiments, one or more of the various other components described above, a solvent or solvent mixture, and a polymerizable component that can form a solid or semisolid removable “mask” when applied to skin.

The polymerizable component in such embodiments may be, for example, polyvinyl alcohol, polyvinyl pyrrolidone, carrageenan, gelatin, dextrin, guar gum, xanthan gum, polyethylene oxide, starch, cellulose derivatives, hydroxyethyl cellulose, ethyl cellulose, carboxymethylcellulose, hydroxypropyl cellulose, polyvinyl alcohol-polyethylene glycol copolymers, methacrylic acid-based copolymers, methacrylate-based copolymers, and the like and combinations thereof. The polymerizable component of such embodiments may be about 15 wt. % to about 50 wt. % of the total composition, and in some embodiments, the polymerizable component may be about 20 wt. % to about 40 wt. % of the total composition or any range or individual concentration encompassed by these example ranges.

The solvent or solvent mixture of such embodiments may include water, ethanol, isopropyl alcohol, ethyl acetate, acetone, glycerol, polyethylene glycol, mineral oil, petrolatum, castor oil, n-methyl pyrrolidone, vegetable oil, honey, oleyl alcohol, dipropylene glycol, saturated polyglycolyzed C8 to C10 glycerides, polyoxyethylated fatty acid glycerides, dimethyl sulfoxide, fatty alcohol, isopropyl myristate, ethyl oleate, essential oils, oleic acid, isosteric acid, fatty acids, and the like and combinations thereof. The solvent or solvent mixture in such embodiments may about 20 wt. % to about 80 wt. % of the total composition or any range or individual concentration encompassed by these example ranges.

The compositions of various embodiments can be in other forms, including topical formulations. Embodiments include, for example, the various components described above incorporated into lotions, foams, liniments, balms, soaps, shampoos, and the like having gradually increasing concentrations of retinoid or retinoid-related compounds.

In some embodiments, the topical formulations can be in the form of a lotion. Lotions are low- to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the topical formulations can be in the form of a foam. Pharmaceutical foams are pressurized dosage forms containing one or more active ingredients that, upon valve actuation, emit a fine dispersion of liquid and/or solid materials in a gaseous medium. Foam formulations are generally easier to apply, are less dense, and spread more easily than other topical dosage forms. Foams may be formulated in various ways to provide emollient or drying functions to the skin, depending on the formulation constituents. Accordingly, this delivery technology is a useful addition to the spectrum of formulations available for topical use.

In some embodiments, the topical formulations can be in the form of a liniment. Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

In some embodiments, the formulations can be in the form of a soap, which are formulations that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerin) is usually liberated and is either left in or washed out and recovered as a useful byproduct, depending on the process employed.

In some embodiments, the topical formulations can be in the form of a shampoo, which is a hair care product used for the removal of oils, dirt, skin particles, dandruff, environmental pollutants, and other contaminant particles that gradually build up in hair. A goal may be to remove the unwanted build-up without stripping out so much sebum as to make hair unmanageable.

Example compositions may include various known components. For example, in some embodiments, the composition may include a solvent such as isopropyl alcohol, benzyl alcohol, dipropylene glycol methyl-ether, butylated hydroxytoluene dipropylene glycol monomethyl-ether, 1-methoxy 2-propanol (glysolv PM/Icinol PM), Ethylene glycol monobutylether (butyl glyxolv/butyl icinol), Butyl di glysolv (butyl-icinol), Transcutol, propylene glycol (PG), N-methyl-2 pyrrolidone (NMP), methylene chloride, diethyl ether, ethanol, acetonitrile, ethyl acetate, benzyl alcohol, a combination of natural oil; ethylene glycol, propylene glycol, dimethyl polysiloxane (DMPX), oleic acid, caprylic acid, 1-octanol, ethanol (denatured or anhydrous), liposomal compositions, suitable plant oils, such as Aloe vera derivatives or sesame seed oil or derivatives thereof, acrylic polymers, rubber-based polymers, polysiloxane-based polymers, polyvinylpyrrolidone-based polymers, dimethyl sulfoxide (DMSO), dimethylformamide (DMF), dimethylacetamide, N-methyl-2-pyrrolidone, hexamethylphosphoramide (HMPA), lecithin, Transfersomes® (bi-component vesicular aggregates), ethosomes, azone, castor oil derivatives, such as ethoxylated castor oil, jojoba oil derivatives, corn oil derivatives, emu oil derivatives, and the like and combinations thereof. The solvent can be present in a concentration of about 5.0% (w/w) to about 15.0% (w/w), about 6.0% (w/w) to about 10.0% (w/w), about 7.5% (w/w) to about 10.5% (w/w), about 8.0% (w/w) to about 10.0% (w/w), or any range or individual concentration of solvent encompassed by these example ranges.

In some embodiments, the compositions may include an antioxidant. Such antioxidant may be, for example, butylated hydroxytoluene, ascorbic acid, ascorbic palmitate, butylated hydroxyanisole, 2,4,5-trihydroxybutyrophenone, 4-hydroxymethyl-2,6-di-tert-butylphenol, erythorbic acid, gum guaiac, propyl gallate, thiodipropionic acid, dilauryl thiodipropionate, tert-butylhydroquinone, tocopherol, and the like and pharmaceutically acceptable salt or ester thereof or combinations thereof. The antioxidant can be present in a concentration of about 0.01% (w/w) to about 1% (w/w) of the total composition or any individual concentration encompassed by this example range.

In some embodiments, the composition may include an emulsifying agent including, for example, various monoglycerides, diglycerides, triglycerides, and blends thereof at a concentration of about 3% (w/w) to about 10% (w/w) of the total composition.

In some embodiments, the compositions may further include a humectant that provides soothing, smoothing, moisturizing, or protects the skin. The humectant is not limited and can be, for example, calamine, dodecylsulphate, sodium lauryl sulphate (SLS), a polyoxyethylene ester of polysorbitan, such as monooleate, monolaurate, monopalmitate, monostearate esters, esters of sorbitan, the polyoxyethylenes ethers, the sodium dioctylsulphosuccinate (DOSS), lecithin, and sodium docusate. The amount of humectant in such compositions may be about 0.01% (w/w) to 5% (w/w) of the total composition.

In some embodiments, the composition may further include an analgesic agent such as, for example, methyl salicylate, codeine, morphine, methadone, pethidine, buprenorphine, hydromorphine, levorphanol, oxycodone, fentanyl, a non-steroidal anti-inflammatory drug (NSAID), and the like and combinations thereof. The amount of the analgesic agent such compositions may be about 0.01% (w/w) to 5% (w/w) of the total composition.

In some embodiments, the compositions may further include a topical debriding agent such as, for example, papain/urea, balsam peru/castor oil/trypsin, chlorophyllin copper complex/papain/urea, collagenase, and the like and combinations thereof. The amount of the debriding agent in such compositions may be about 0.01% (w/w) to 5% (w/w) of the total composition.

In some embodiments, the compositions may further include a topical emollient such as, for example, urea, ammonium lactate, salicylic acid/urea, vitamins A, D, and E, ammonium lactate/pramoxine, vitamin A & D, dexpanthenol, ammonium lactate/urea, salicylic acid/urea, aloe vera, lanolin, and the like and combinations thereof. The amount of the emollient such compositions may be about 0.01% (w/w) to 5% (w/w) of the total composition.

A cream base may be prepared by conventional techniques well known to those skilled in the art. Generally, a suitable process includes admixing the various ingredients of the cream in appropriate relative amounts in any order that is convenient and thereafter, if necessary adjusting the pH to the final desired value. For example, the components of the base may be mixed together at a temperature of about 65° C. to about 75° C. until an emulsion has formed, and a therapeutic agent may be added after cooling the emulsified cream base or during mixing.

As is known in the art, certain means for administering may require the use of particular components of the formulation. Such components are described above and can be appropriately incorporated into the compositions. In particular embodiments, administering may be carried out by applying the composition to the skin of a patient. Such administering can be carried out by hand or using an applicator or appliance and administering can be carried out by another person or the person to which the composition is administered.

Further embodiments are directed to the individual compositions described above, and kits containing two or more of the compositions described above. Such kits may include two or more containers containing compositions with increasing concentrations of retinoid or retinoid-related compound. In some embodiments, at least one of the two or more containers may include a composition containing a maximum concentration of retinoid or retinoid-related compound, which may be about 0.8 wt. % to about 1.5 wt. % retinoid or retinoid-related compound. In some embodiments, the kits may contain at least a second container that may include compositions having a retinoid or retinoid-related compound concentration of about 10%, about 25%, about 50%, about 75% of the maximum concentration or any individual percentage encompassed by these example concentrations. In particular embodiments, the kits may include two or more individual containers containing compositions having retinoid or retinoid-related compound concentrations of about 5% to about 25% of a maximum concentration, about 30% to about 50% of a maximum, about 70% to about 90% of a maximum concentration, about 100% of a maximum concentration, or any individual percentage encompassed by these example concentrations.

In certain embodiments, the kits may further include instructions for administering each of the two or more compositions. For example, such compositions may include instructions for administering each of the two or more compositions, daily for one to 14 days, weekly for 1 to 4 or 6 weeks, or monthly for 1 to 4 or 6 months. In other embodiments, the instructions may include instructions for administering each of the two or more compositions until the patient is substantially free from side effects such as redness, swelling, and itching of the skin to which the compositions are applied or combinations thereof.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

Various compositions will be made as outlined in Table 1 and sequentially administered to patients to provide exfoliation of the skin without redness, swelling, and itching of the skin generally associated with retinoid or retinoid-related compound administration.

| | Retinoid | Hyaluric acid | Niacin | Dexpanthenol |
|---|---|---|---|---|
| Composition 1 | | | | |
| Ex. 1 | 0.25 | | | |
| Ex. 2 | 0.25 | 0.50 | | |
| Ex. 3 | 0.25 | | 0.50 | |
| Ex. 4 | 0.30 | | | 0.50 |
| Ex. 5 | 0.30 | 0.50 | 0.50 | |
| Ex. 6 | 0.30 | | 0.50 | 0.50 |
| Composition 2 | | | | |
| Ex. 7 | 0.50 | | | |
| Ex. 8 | 0.50 | 0.50 | | |
| Ex. 9 | 0.50 | | 0.50 | |
| Ex. 10 | 0.50 | | | 0.50 |
| Ex. 11 | 0.75 | 0.50 | 0.50 | |
| Ex. 12 | 0.75 | | 0.50 | 0.50 |
| Composition 3 | | | | |
| Ex. 13 | 0.75 | | | |
| Ex. 14 | 0.75 | 0.50 | | |
| Ex. 15 | 0.75 | | 0.50 | |
| Ex. 16 | 0.75 | | | 0.50 |
| Ex. 17 | 1.30 | 0.50 | 0.50 | |
| Ex. 18 | 1.30 | | 0.50 | 0.50 |
| Composition 4 | | | | |
| Ex. 19 | 1.00 | | | |
| Ex. 20 | 1.00 | 0.50 | | |
| Ex. 21 | 1.00 | | 0.50 | |
| Ex. 22 | 1.50 | | | 0.50 |
| Ex. 23 | 1.50 | 0.50 | 0.50 | |
| Ex. 24 | 1.50 | | 0.50 | 0.50 |

1. A method for treating a skin condition, comprising:
topically administering to a subject in need of treatment one or more compositions containing less than a maximum concentration of retinoid or retinoid-related compound for one or more days; and
topically administrating to the subject one or more compositions containing a maximum concentration of retinoid or retinoid-related compound after topically administering to a subject in need of treatment one or more compositions containing less than a maximum concentration of retinoid or retinoid-related compound for one or more days.

2. The method of claim 1, wherein the maximum concentration retinoid or retinoid-related compound about 0.8 wt. % to about 1.5 wt. %.

3. The method of claim 1, wherein the retinoid or retinoid-related compound is selected from the group consisting of retinol (vitamin A), retinal, retiferol, tretinoin, all-trans-retinoic acid, retinoic acid, Retin-A, isotretinoin, alitretinoin (9-cis-retinoic acid), etretinate, acitretin, tazarotene, bexarotene, Adapalene, and combinations thereof

4. The method of claim 1, wherein the wherein the each of the compositions further comprise a base selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, liposomal base, and combinations thereof.

5. The method of claim 4, wherein the base comprises about 45% (w/w) to about 99.75% (w/w) of the total composition.

6. The method of claim 1, wherein the skin condition is selected from the group consisting of acne, photoaging, psoriasis, ichthyosis, hair loss, and tumors.

7. A method for treating skin condition comprising:
topically administering to a subject in need of treatment a first course of treatment comprising daily administration of a composition containing first composition containing 0.08 wt. % to about 0.5 wt. % retinoid or retinoid-related compound; and
topically administering to the subject a second course of treatment comprising daily administration of a second composition containing about 0.3 wt. % to about 0.8 wt. % retinoid or retinoid-related compound after the first course of treatment is completed, wherein the second composition has a higher concentration of retinoid or retinoid-related compound than the first composition.

8. The method of claim 7, wherein the first course of treatment lasts 1 to 14 days.

9. The method of claim 7, wherein the second course of treatment lasts 1 to 14 days.

10. The method of claim 7, wherein the retinoid or retinoid-related compound is selected from the group consisting of retinol (vitamin A), retinal, retiferol, tretinoin, all-trans-retinoic acid, retinoic acid, Retin-A, isotretinoin, alitretinoin (9-cis-retinoic acid), etretinate, acitretin, tazarotene, bexarotene, Adapalene, and combinations thereof.

11. The method of claim 7, wherein the each of the first and second compositions further comprise a base selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, liposomal base, and combinations thereof.

12. The method of claim 7, further topically administering to the subject a third course of treatment comprising daily administration of a third composition containing about 0.5 wt. % to about 1.5 wt. % retinoid or retinoid-related compound after the second course of treatment is completed, wherein the third composition has a higher concentration of retinoid or retinoid-related compound than the second composition.

13. The method of claim 12, wherein the third course of treatment lasts 1 to 14 days.

14. The method of claim 12, wherein the retinoid or retinoid-related compound is selected from the group consisting of retinol (vitamin A), retinal, retiferol, tretinoin, all-trans-retinoic acid, retinoic acid, Retin-A, isotretinoin, alitretinoin (9-cis-retinoic acid), etretinate, acitretin, tazarotene, bexarotene, Adapalene, and combinations thereof.

15. The method of claim 12, wherein the third composition further comprises a base selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, liposomal base, and combinations thereof.

16. The method of claim 12, further topically administering to the subject a fourth course of treatment comprising daily administration of a fourth composition containing about 0.7 wt. % to about 1.5 wt. % retinoid or retinoid-related compound after the third course of treatment is completed, wherein the fourth composition has a higher concentration of retinoid or retinoid-related compound than the second composition.

17. The method of claim 16, wherein the fourth course of treatment lasts 1 to 14 days.

18. The method of claim 16, wherein the retinoid or retinoid-related compound is selected from the group consisting of retinol (vitamin A), retinal, retiferol, tretinoin, all-trans-retinoic acid, retinoic acid, Retin-A, isotretinoin, alitretinoin (9-cis-retinoic acid), etretinate, acitretin, tazarotene, bexarotene, Adapalene, and combinations thereof.

19. The method of claim 16, wherein the fourth compositions further comprise a base selected from the group consisting of white petrolatum, white petrolatum USP, mineral jelly, petroleum jelly, yellow petrolatum, yellow soft paraffin, white soft paraffin, fats, waxes, sterols, fat-soluble vitamins, monoglycerides, diglycerides, triglycerides, phospholipids, PCCA plasticized base, liposomal base, and combinations thereof.

20. The method of claim 7, wherein the skin condition is selected from the group consisting of acne, photoaging, psoriasis, ichthyosis, hair loss, and tumors.
